(19) United States
(12) Patent Application Publication
(10) Pub. No.: US 2007/0248997 A1
(43) Pub. Date: Oct. 25, 2007

(54) METHOD OF USING ONE-DIMENSIONAL AND MULTI-DIMENSIONAL NUCLEAR MAGNETIC RESONANCE TO IDENTIFY COMPOUNDS THAT INTERACT WITH TARGET BIOMOLECULES

(75) Inventor: JACQUES BRIAND, Collegeville, PA (US)

Correspondence Address: GLAXOSMITHKLINE;Corporate Intellectual Property - UW2220, P.O. Box 1539, King of Prussia, PA, 19406-0939, US

(73) Assignee: Smithkline Beecham Corporation

(21) Appl. No.: 11/745,780

(22) Filed: May 8, 2007

Related U.S. Application Data
(62) Division of application No. 10/070,128, filed on Feb. 27, 2002.
(60) Provisional application No. 60/156,557, filed on Sep. 29, 1999.

(51) Int. Cl. G01N 33/573

(52) U.S. Cl. 435/7.8

(57) ABSTRACT

The invention provides a method using one-dimensional and multi-dimensional NMR spectroscopy for identifying ligands to target biomolecules.

This application is a divisional application of U.S. Ser. No. 10/070,128 filed Feb. 27, 2002, which claims priority to PCT/US00/26949, which has an international filing date of 29 Sep. 2000 and claims priority to U.S. Provisional Application 60/156,557 filed on 29 Sep. 1999.

### FIELD OF THE INVENTION

The present invention relates to a method for identifying and classifying compounds that bind to target biomolecules using one- or two-dimensional nuclear magnetic resonance (NMR) spectroscopy.

### BACKGROUND OF THE INVENTION

The use of nuclear magnetic resonance for screening and detecting weakly binding compounds to biomolecular targets has been recently described in patent documents U.S. Pat. No. 5698401, U.S. Pat. No. 5804390, U.S. Pat. No. 5891643, DE19649359, PCT publications WO 98/48264, WO 98/57155 and other publications, Meyer et al. Eur. J. Biochem. 246 705-709 (1997), Lin et al. J. Am. Chem. Soc. 119 5249-5250 (1997), Chen et al. J. Am. Chem. Soc. 120 10258-10259 (1998), Lin et al. J. Org. Chem. 62 8930-8931 (1997), Hajduk et al. J. Am. Chem. Soc. 119 12257-12261 (1997), Shuker et al. Science 274 1531-1537 (1996), Klein et al. J. Am. Chem. Soc. 121 5336-5337 (1999), Mayer et al. Angew. Chem. (1999). There are several NMR techniques covered in these publications and they all rely at various degrees on observing changes in NMR chemical shifts, changes in resonance linewidths, changes in translational diffusion, or changes in dipolar relaxation phenomena of either the target or the ligand upon undergoing exchange between the bound and unbound state. The bound and unbound populations are dictated by the affinity between the target molecule (E) and the ligand (L) following the binding reaction at equilibrium
E+L [symbol] EL
where K_{d} is the dissociation constant expressed by

$${K}_{d} = \frac{[ E ] [ L ]}{[ EL ]}$$

The bound (p_{b}) and unbound (pᵤ) populations are expressed are by

$${p}_{b} = \frac{[ L ]}{{K}_{d} + [ L ]} \qquad {p}_{u} = \frac{{K}_{d}}{{K}_{d} + [ L ]}$$

The greater the value of K_{d}, the smaller the affinity. It is well known in the art that if binding affinity is weak (K_{d} typically at sub-millimolar concentration or higher), the bound population can be small and at typical NMR ligand concentrations (submillimolar to millimolar), a relatively large target concentration (typically up to low submillimolar) is required for populating the bound state and observe perturbations on the NMR signal of the ligand. Inversely, if the NMR signal of the target molecule is being observed, a large ligand concentration is required for populating the target molecule and a relatively large concentration (typically submillimolar range) of the target molecule is also required in order to record a NMR spectrum with a reasonable S/N ratio. The latter also has the shortfall of being limited to relatively low molecular weight targets (<20-35 KDa) and requires isotopic labelling of the target. Thus, a major shortcoming of these existing NMR screening techniques for identifying weakly binding ligands is their necessity of having to use a high target concentration in order to perturb the NMR spectral parameters at detectable levels.

Clearly, there exists a need for a faster and less expensive method for identifying ligands to target biomolecules as this undergirds the pharmaceutical discovery process, among other things. Further, these methods have a present benefit of, among other things, being useful to screen compounds for biological activities. Such methods are also useful for determining the role of targets and such compounds in pathogenesis of infection, dysfunction and disease in individuals. There is also a need for methods for identification and characterization of such targets and their antagonists and agonists to find ways to prevent, ameliorate or correct such infection, dysfunction and disease in individuals. The present fulfils this unmet need by providing methods using one-dimensional and multi-dimensional NMR spectroscopy for identifying compounds that interact with target biomolecules.

### SUMMARY OF THE INVENTION

The present invention relates to methods using one-dimensional and multi-dimensional NMR spectroscopy for identifying ligands to target biomolecules. One-dimensional and two-dimensional NMR spectroscopy are preferred in the methods of the invention. Moreover, methods are provided by the invention for using one-dimensional and multi-dimensional nuclear magnetic resonance to identify products, ligands or substrates of enzymatic target biomolecules.

A further embodiment of the invention provides a method for identifying compounds that bind to or interact with a specific target molecule. One preferred method comprises the steps of: a) mixing a substrate or product of a target biomolecule with one or a mixture of chemical compounds; b) generating a first one- or two-dimensional spectrum that displays either 1H,3H, 11B,13C,15N,19F,29S or 31P chemical shifts in the first dimension or 1H,3H,11B,13C,15N,19F,29S or 31P chemical shifts in the other dimension of substrate or product in step a); c) exposing substrate or product and mixture of chemical compounds in step a) to a target molecule for one or more incubation times; d) generating a second first one- or two-dimensional spectrum that displays either 1H,3H, 11B,13C,15N,19F,29S or 31P chemical shifts in the first dimension or 1H,3H,11B,13C,15N,19F,29S or 31P chemical shifts in the other dimension of substrate or product in step a) that has been exposed to the target molecule in step c) in the presence of one or mixture of chemical compounds in step a); e) comparing said first and second one- or two-dimensional NMR spectra after one or more said incubation times in step c) to determine differences between said first and second NMR spectra, the differences observed along either or both chemical shift dimensions identifying the transformation of said substrate classifying the presence of one or more compounds that are ligands which have bound to the enzymatic site of said target molecule.

Another embodiment of the invention is a method of identifying compounds that interact with a target molecule comprising the steps of: a) mixing a substrate, product or ligand of a target with at least one chemical compounds; b) generating a first spectrum that displays either a chemical shift in the first dimension or a chemical shifts in the other dimension of substrate, product or ligand in step a); c) exposing substrate, product or ligand and mixture of chemical compounds in step a) to a target molecule for one or more incubation times; d) generating a second spectrum that displays either a chemical shifts in the first dimension or a chemical shifts in the other dimension of substrate or product in step a) that has been exposed to the target molecule in step c) in the presence of one or mixture of chemical compounds in step a); e) comparing said first spectrum and second spectrum after one or more said incubation times in step c) to determine at least one difference between said first spectrum and second spectrum, the differences observed along either or both chemical shift dimensions identifying the transformation of said substrate and classifying the presence of one or more compounds that are substrates, products or ligands that interact with said target molecule.

Still another embodiment of the invention provides a method of identifying compounds that interact with a target molecule comprising the steps of: a) exposing substrate to a target molecule for one or more incubation times; b) generating one or more spectra at one or more incubation times of said substrate and said target molecule of step a), c) exposing said substrate and one or mixture of chemical compounds for one or more incubation times; d) generating one or more spectra at one or more incubation times of said substrate, said target molecule and said compounds of step c); e) comparing at least one spectrum of step b) with at least one spectrum of step d) to determine at least one difference between said spectrum of step b) with said spectrum of step d), the differences observed along either or both chemical shift dimensions identifying the transformation of said substrate and classifying the presence of one or more compounds that are substrates, products or ligands that interact with said target molecule.

The invention also provides a method wherein step a) further comprises a target that is a biomolecule.

The invention still further provides a method wherein step a) further comprises a chemical compound that is in solution or attached to a solid substrate or matrix.

Another embodiment of the invention is a method wherein step b) further comprises a first spectrum selected from the group consisting of a one-dimensional, two-dimensional or three-dimensional spectrum, and/or wherein said first spectrum displays a chemical shift in said first dimension selected from the group consisting of a 1H,3H,11B,13C,15N,19F,29S or 31P chemical shift, and a chemical shift in said other dimension selected from the group consisting of 1H,3H,11B,13C,15N,19F,29S or 31P chemical shift.

A further embodiment of the invention provides a method wherein said exposing step of step c) further comprises a mixture comprising between 2 and 100 chemical compounds, and/or wherein said incubation times number between 1 and 20, 30, 40 50 or greater.

A still further embodiment of the invention provides a method of claim 1 wherein step d) said second spectrum displays a chemical shift in said first dimension selected from the group consisting of a 1H,3H,11B, 13C, 15N, 19F,29S or 31 P chemical shift, and a chemical shift in said other dimension selected from the group consisting of 1H,3H,11B,13C,15N,19F,29S or 31P chemical shift.

Also provided by the invention is a method wherein said comparing step of step e) comprises comparing first one- or two-dimensional NMR spectra and second one- or two-dimensional NMR spectra after one or more said incubation times.

A method is provided wherein the determining step of step e) comprises a method selected from the group consisting of an algorithm, a computer algorithm, and visual inspection.

Another method of the invention provides a method wherein an interaction is selected from the group consisting of molecule-molecule binding, ligands bound to an enzymatic site of said target molecule.

A preferred embodiment is a method of determining an interaction constant (α) comprising the steps of: a) exposing a substrate or product to a target molecule for one or more incubation times; b) generating a first spectrum that displays either a chemical shift in the first dimension or a chemical shifts in an other dimension of the substrate or product in step a) that has been exposed to the target molecule; c) mixing a substrate or product with the first ligand; d) exposing the substrate or product and the first ligand to the target molecule for one or more incubation times; e) generating a second spectrum that displays either a chemical shift in the first dimension or a chemical shift in the other dimension of substrate or product in step c) that has been exposed to the target molecule in step d) in the presence of the first ligand in step c); f) mixing the substrate or product with one or more chemical compounds; g) exposing the substrate or product and one or more chemical compounds to a target molecule for one or more incubation times; h) generating a third spectrum that displays either a chemical shift in the first dimension or a chemical shift in the other dimension of substrate or product in step f) that has been exposed to the target molecule in step g) in the presence of the one or more chemical compounds in step f); i) mixing substrate or product with first ligand and one or more chemical compounds; j) exposing the substrate or product, the first ligand and the one or more chemical compounds to the target molecule for one or more incubation times; k) generating a fourth spectrum that displays either a chemical shift in the first dimension or a chemical shift in the other dimension of the substrate or product in step i) that has been exposed to the target molecule in step j) in the presence of the first ligand and the one or more chemical compounds in step f); l) determining a conversion rate or conversion rates of each substrate or product from each spectrum of steps b), e), h) and k); and deriving an interaction constant (α) from a steady-state rate equation.

A method using NMR for screening for ligands which exhibit synergistic effects on a target in the presence of another ligand is also provided by the invention.

A method wherein the target is an enzyme comprising more than two binding sites is further provided, including, for example a binding site comprising a proximal and/or integral sub-binding site or sites.

Also provided is a method wherein the two binding sites are a substrate- and co-enzyme-binding site.

A method wherein the rate is determined by the following equation:

$$v = {V}_{m} / [ 1 + ( \frac{{K}_{M}}{S} ) ( 1 + \frac{{I}_{1}}{{K}_{EI 1}} + \frac{{I}_{2}}{{K}_{EI 2}} + \frac{{I}_{1} {I}_{2}}{α {K}_{EI 1} {K}_{EI 2}} ) ]$$

wherein S, I₁ and I₂ are the substrate, inhibitor I₁ and inhibitor I₂ concentrations, respectively.

Further methods are provided wherein said at least one chemical compound is provided in a multiwell vessel loaded with target and ligand and/or substrate and/or product.

Methods are also provided wherein a target-substrate reaction is quenched at a selected time to stop the reaction.

Various changes and modifications within the spirit and scope of the disclosed invention will become readily apparent to those skilled in the art from reading the following descriptions and from reading the other parts of the present disclosure.

### BRIEF DESCRIPTION OF THE DRAWINGS

FIG. 1 depicts the deformylation of For-Met-Ala-Ser-OH catalyzed by the Ni²⁺ form of Peptide Deformylase (S. Aureus) (herein also referred to as “PDF”) over a range of incubation times, as monitored by 1H nuclear magnetic resonance spectroscopy. The top row is a control experiment depicting the stability of For-Met-Ala-Ser-OH. Only the resonance peak of the amide proton of the Ser residue is shown over the time course. In the middle row, For-Met-Ala-Ser-OH (S: substrate) is deformylated by Ni-PDF and its transformation into Met-Ala-Ser-OH (P: product) is monitored through the appearance of a second resonance peak, labelled as “P” at the left of the original resonance peak labelled as “S”. In the bottom row, 8-hydroxyquinoline is added to the mixture and the deformylation of For-Met-Ala-Ser-OH is slightly inhibited, as seen by the decreased rate at which the resonance peak “P” increases over the time course.

Staphylococcus aureus Peptide Deformylase is described in U.S. patent application Ser. No. 08/911,844, filed Aug. 15, 1997, and European Patent Applications EP008798879A2 and EP00879879A3. The methods of the example could also be adapted for use with Streptococcus pneumoniae pdf1 (U.S. patent application Ser. No. 08/991,023, filed Dec. 15, 1997 and European Patent Applications EP00863152A3 and EP00863152A2) or Streptococcus pneumoniae pdf2 (U.S. patent application Ser. No. 08/989,558, filed Dec. 12, 1997 and European Patent Application EP00863205A1).

FIG. 2 follows the same description as FIG. 1 with the only difference being that the resonance peak of the formate group is monitored over the same time course. Formate is a by-product of the deformylation of For-Met-Ala-Ser-OH.

FIG. 3 depicts the reaction scheme when two substrate-competitive inhibitors (I₁ and I₂) and a substrate (S) react with an enzyme (E). It is assumed that I₁I₂, I₁S, I₂S, I₁I₂S, EI₁S, EI₂S and EI₁I₂S are not formed in this system. An interaction constant α between I₁ and I₂ in the EI₁I₂ complex has the following meaning: when I₁ and I₂ interact with the same site of E, they prevent each other from binding to E, i.e. I₁I₂ is not formed and thus α=∞. If I₁ and I₂ interact with different sites of E, ∞>α>0. When the interactions of I₁ and I₂ with E are strictly independent of each other, α=1. When a positive attraction occurs or if synergy exists between the two inhibitors in the EI₁I₂ complex, 1>α>0. When I₁ and I₂ interact repulsively in the EI₁I₂ complex, ∞>α>1.

FIG. 4 depicts the deformylation of For-Met-Ala-Ser-OH catalyzed by the Ni²⁺ form of Peptide Deformylase (S.Aureus) over a range of incubation times, as monitored by 1H nuclear magnetic resonance spectroscopy of the buildup of the formate (For) moiety. Four slopes are illustrated in this figure, the first one (∘) is a “control” experiment using 2.5 mM of substrate (For-Met-Ala-Ser-OH) and 1 uM of enzyme. The second slope (□) contains 0.4 mM of 8-hydroxyquinoline (I₁) in the presence of 2.5 mM of substrate and 1 uM of enzyme. The third slope (∇) contains 0.15 mM of N-Nitro-N′-(N-phenylamino)guanidine (I₂) in the presence of 2.5 mM of substrate and 1 uM of enzyme. Finally the fourth slope (⋄) contains both I₁ and I₂ at 0.4 mM and 0.15 mM, respectively, in the presence of 2.5 mM of substrate and 1 uM of enzyme. Calculating the four slopes and using the steady-state rate equations for this system, it is straightforward to calculate the K_{EI1}, K_{EI2}, and α value. Assuming a K_{M} of 2.5 mM, we get 335 uM, 151 uM and 0.72, for K_{EI1}, K_{EI2}, and α, respectively. This example demonstrates that it is possible to screen for a second inhibitor I₂ in the presence of a first inhibitor I₁ and determine whether they interact in a highly desirable synergistic fashion, or whether they interact repulsively or compete for the same site.

FIG. 5 depicts the deformylation of For-Met-Ala-Ser-OH catalyzed by the Ni²⁺ form of Peptide Deformylase (S.Aureus) over a range of incubation times, as monitored by 1H nuclear magnetic resonance spectroscopy. Only the resonance peak of the amide proton of the Ser residue is shown over the time course. In the top row, For-Met-Ala-Ser-OH (S: substrate) is deformylated by Ni-PDF and its transformation into Met-Ala-Ser-OH (P: product) is monitored through the appearance of a second resonance peak, labelled as “P” at the left of the original resonance peak labelled as “S”. In the bottom row, the same experiment as in the top row is performed but at an incubation time of about 22-24 minutes, the enzymatic reaction is quenched by adding hydrazine (15 mM final sample concentration) to the sample. Following this procedure, the deformylation of For-Met-Ala-Ser-OH is further monitored by 1 H nuclear magnetic resonance spectrospcopy over a time course and it can be seen that the enzymatic reaction has been successfully stopped, as judged from the lack of progression of the resonance peaks labelled as “S” and “P”.

FIG. 6 illustrates further examples demonstrating that it is possible to screen for a second inhibitor I₂ in the presence of a first inhibitor I₁ and determine whether they interact in a highly desirable synergistic fashion, or whether they interact repulsively or compete for the same site. In the specific examples shown here, the Yonetani-Theorell graphical method is used for determining the interaction constant “α” between two inhibitors. In this method, enzyme kinetic rates, ν, are measured at fixed concentrations of substrate and in the presence of variable concentrations of two inhibitors I₁ and I₂. First, a set of lines is obtained for 1/ν versus [I₁] for a series of fixed [I₂]. The abscissa at the point of intersection of this set of lines corresponds to −αK_{EI1}, where K_{EI1} is the constant of inhibition for inhibitor I₁. K_{EI1} is also graphically determined using the Dixon graphical method: a family of lines for 1/ν versus [I₁] for a series of fixed [S] is obtained and the abscissa at the point of intersection of this family of lines corresponds to −K_{EI1}. In a) and b), two pairs of ligands are shown to interact synergistically with α<1, suggesting that they can bind simultaneously in a synergistic fashion. Inversely in c), formohydroxamic acid and 8-hydroxyquinoline are shown to compete for the same subsite with α>>1, suggesting that they interact repulsively and cannot bind simultaneously with the enzyme.

### DESCRIPTION OF THE INVENTION

An embodiment of the present invention provides a target molecule involved in a catalytic reaction of a substrate into a final product. Not being limited in any way by a theoretical or mathematical model, a simple example of such a process may be described, among other ways, by the following reaction mechanism
E+S [symbol] ES [symbol] E+P
where the target molecule is an enzyme (E) which catalyzes the conversion of a substrate “S” into a product “P”. The velocity of the enzyme-catalyzed reaction, ν, can be expressed as a function of substrate and enzyme concentration by the well-known Michaelis-Menten equation

$$v = \frac{{k}_{cat} [ S ] [ {E}_{0} ]}{{K}_{M} + [ S ]}$$

where [E₀] is the total enzyme concentration, [S] is the substrate concentration and the ratio k_{cat}/K_{M} is a measure of the catalytic efficiency of the enzyme for a particular substrate. It is well-known in the art of enzymology, that, although there can be a wide range of catalytic efficiencies for various substrates of a particular enzyme, this catalytic efficiency can be extreme under favorable condition up to diffusion limits of about 10⁹s⁻¹M⁻¹.

In a preferred embodiment of the present invention, this catalytic efficiency is exploited in order to decrease by several orders of magnitude the amount of target molecule required to detect binding of a compound to the target molecule. This can be done by monitoring changes in the conversion rate of a substrate into a product when the compound is present.

Another preferred embodiment provides a method wherein changes observed in a parameter of an enzymatic reaction, such as reaction rate can be measured. Example 1 provides on such method. For target enzymes having catalytic efficiencies in the range of 10⁵s⁻¹M⁻¹ for that substrate, it is preferable to use a target concentration which is at least two orders of magnitude smaller than the concentration that would have been required for identifying a known substrate as a ligand by any known NMR technique, such as the well-known technique of differential line broadening.

Another advantage of the methods of the invention for weakly binding ligands, is that an NMR spectrum also contains their fingerprint which can be used to monitor their stability simultaneously with the substrate conversion.

Another advantage compared to coupled assays is to reduce the number of false positives found in coupled assays which rely on additional reagents which can potentially react with the screened compounds downstream or upstream and appear as if the compound was interacting with the target of interest. In the present invention, there is no need for these additional reagents since the substrate is directly observed by NMR.

A further embodiment of the invention provides method for identifying compounds that bind to or interact with a specific target molecule. One preferred method comprises the steps of: a) mixing a substrate or product of a target biomolecule with one or a mixture of chemical compounds; b) generating a first one- or two-dimensional spectrum that displays either 1H,3H,11B,13C,15N,19F,29S or 31P chemical shifts in the first dimension 1H,3H,11B,13C,15N,19F,29S or 31P chemical shifts in the other dimension of substrate or product in step a); c) exposing substrate or product and mixture of chemical compounds in step a) to a target molecule for one or more incubation times; d) generating a second first one- or two-dimensional spectrum that displays either 1H,3H, 11B,13C,15N,19F,29S or 31 P chemical shifts in the first dimension or 1H,3H,11B,13C,15N,19F,29S or 31P chemical shifts in the other dimension of substrate or product in step a) that has been exposed to the target molecule in step c) in the presence of one or mixture of chemical compounds in step a); e) comparing said first and second one- or two-dimensional NMR spectra after one or more said incubation times in step c) to determine differences between said first and second NMR spectra, the differences observed along either or both chemical shift dimensions identifying the transformation of said substrate or product and classifying the presence of one or more compounds that are ligands which have bound to the enzymatic site of said target molecule.

Another embodiment of the invention is a method of identifying compounds that interact with a target molecule comprising the steps of: a) mixing a substrate, product or ligand of a target with at least one chemical compounds; b) generating a first spectrum that displays either a chemical shift in the first dimension or a chemical shifts in the other dimension of substrate, product or ligand in step a); c) exposing substrate, product or ligand and mixture of chemical compounds in step a) to a target molecule for one or more incubation times; d) generating a second spectrum that displays either a chemical shifts in the first dimension or a chemical shifts in the other dimension of substrate or product in step a) that has been exposed to the target molecule in step c) in the presence of one or mixture of chemical compounds in step a); e) comparing said first spectrum and second spectrum after one or more said incubation times in step c) to determine at least one difference between said first spectrum and second spectrum, the differences observed along either or both chemical shift dimensions identifying the transformation of said substrate and classifying the presence of one or more compounds that are substrates, products or ligands that interact with said target molecule.

The invention also provides a method wherein step a) further comprises a target that is a biomolecule.

The invention still further provides a method wherein step a) further comprises a chemical compound that is in solution or attached to a solid substrate or matrix.

Another embodiment of the invention is a method wherein step b) further comprises a first spectrum selected from the group consisting of a one-dimensional, two-dimensional or three-dimensional spectrum, and/or wherein said first spectrum displays a chemical shift in said first dimension selected from the group consisting of 1H,3H,11B,13C,15N,19F,29S or 31P chemical shift, and a chemical shift in said other dimension selected from the group consisting of 1H,3H,11B,13C,15N,19F,29S or 31P chemical shift.

A further embodiment of the invention provides a method wherein said exposing step of step c) further comprises a mixture comprising between 2 and 100 chemical compounds, and/or wherein said incubation times number between 1 and 20, 30, 40, 50 or greater.

A still further embodiment of the invention provides a method of claim 1 wherein step d) said second spectrum displays a chemical shift in said first dimension selected from the group consisting of a 1H,3H,11B,13C,15N,19F,29S or 31 P chemical shift, and a chemical shift in said other dimension selected from the group consisting 1H,3H,11B,13C,15N,19F,29S or 31P chemical shift.

Also provided by the invention is a method of wherein said comparing step of step e) comprises comparing first one- or two-dimensional NMR spectra and second one- or two-dimensional NMR spectra after one or more said incubation times.

A method is provided wherein the determining step of step e) comprises a method selected from the group consisting of an algorithm, a computer algorithm, and visual inspection.

Another method of the invention provides a method wherein an interaction is selected from the group consisting of molecule-molecule binding, ligands bound to an enzymatic site of said target molecule.

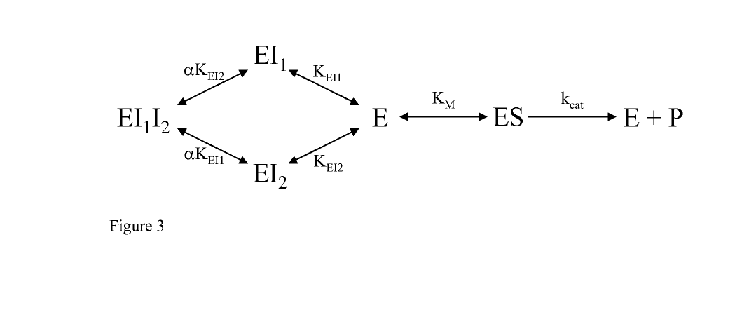

In a further embodiment of this invention, it can be used in the presence of a first ligand for screening for ligands which exhibit highly desirable synergistic effects with the first ligand. Typical applications are for screening for ligands of enzymes which have more than two binding sites (substrate- and co-enzyme-binding sites, for instance) or ligands of proximal subsites on an enzyme with only one active center. FIG. 3 depicts the reaction scheme when two substrate-competitive inhibitors (I₁ and I₂) and a substrate (S) react with an enzyme (E). This is well-known in the art of enzymology and was first demonstrated by Slater and Bonner, Biochem. J. 52 185 (1952). The initial steady-state rate equation for this system is expressed by:

$$v = {V}_{m} / [ 1 + ( \frac{{K}_{M}}{S} ) ( 1 + \frac{{I}_{1}}{{K}_{EI 1}} + \frac{{I}_{2}}{{K}_{EI 2}} + \frac{{I}_{1} {I}_{2}}{α {K}_{EI 1} {K}_{EI 2}} ) ]$$

where S, I₁ and I₂ are the substrate, inhibitor I₁ and inhibitor I₂ concentrations, respectively. An interaction constant α between I₁ and I₂ in the EI₁I₂ complex has the following meaning: when I₁ and I₂ interact with the same site of E, they prevent each other from binding to E, i.e. EI₁I₂ is not formed and thus α=∞. If I₁ and I₂ interact with different sites of E, ∞>α>0. When the interactions of I₁ and I₂ with E are strictly independent of each other, α=1. When a positive attraction occurs or if synergy exists between the two inhibitors in the EI₁I₂ complex, 1>α>0. When I₁ and I₂ interact repulsively in the EI₁I₂ complex, ∞>α>1. A number of causes may be involved in this interaction constant α, such as ion-dipole, interionic, interdipole, hydrophobic, and hydrophilic interactions, as well a steric hindrance and protein conformational changes. For screening for ligands in proximal subsites, this system is particularly useful since it provides a way to determine whether two ligands in proximal subsites form a suitable pair of ligands which can then be chemically combined to span the active site and increase affinity, as described in patent documents U.S. Pat. No. 5698401, U.S. Pat. No. 5804390, U.S. Pat. No. 5891643. For screening purposes in the presence of a first ligand, the interaction constant “α” serves merely to identify ligands that may potentially exhibit desirable synergistic effects with the first ligand. A preferred embodiment of the invention is based on a model that assumes that the inhibitors are substrate-competitive inhibitors. However, the modes of inhibition of the identified pairs of ligands are not necessarily known initially, and should not be deemed to be in any way limitative of the scope of the invention. Therefore, it is usually desirable to determine their mode of inhibition by standard enzymatic methods, such as the Lineweaver-Burks method, among others, in order to further characterize them and ascertain their relevance in a given situation. The skilled artisan will understand what method to use in a given situation in light of the disclosure herein and the state of the art. One preferred embodiment of this method comprising the steps of: a) exposing a substrate to a target molecule for one or more incubation times; b) generating a first spectrum that displays either a chemical shift in the first dimension or a chemical shifts in an other dimension of the substrate or product in step a) that has been exposed to the target molecule; c) mixing a substrate or product with the first ligand; d) exposing the substrate or product and the first ligand to the target molecule for one or more incubation times; e) generating a second spectrum that displays either a chemical shift in the first dimension or a chemical shift in the other dimension of substrate or product in step c) that has been exposed to the target molecule in step d) in the presence of the first ligand in step c); f) mixing the substrate or product with one or more chemical compounds; g) exposing the substrate or product and one or more chemical compounds to a target molecule for one or more incubation times; h) generating a third spectrum that displays either a chemical shift in the first dimension or a chemical shift in the other dimension of substrate or product in step f) that has been exposed to the target molecule in step g) in the presence of the one or more chemical compounds in step f); i) mixing substrate or product with first ligand and one or more chemical compounds; j) exposing the substrate or product, the first ligand and the one or more chemical compounds to the target molecule for one or more incubation times; k) generating a fourth spectrum that displays either a chemical shift in the first dimension or a chemical shift in the other dimension of the substrate or product in step i) that has been exposed to the target molecule in step j) in the presence of the first ligand and the one or more chemical compounds in step f); l) determining a conversion rate or conversion rates of each substrate or product from each spectrum of steps b), e), h) and k); and deriving an interaction constant (α) from a steady-state rate equation.

Targets, products, ligands and substrates of the invention may be polypeptides and/or polynucleotides. These compounds may be natural substrates, products and ligands or may be structural or functional mimetics. See, e.g., Coligan et al., Current Protocols in Immunology 1(2): Chapter 5 (1991).

Moreover, these compounds may be polypeptides and polynucleotides responsible for many biological functions, including many disease states form which an individual may suffer, in particular human diseases. It is therefore desirable to devise screening methods to identify compounds which stimulate or which inhibit the function of a polypeptide or polynucleotide, and such methods are provided herein. Compounds may be identified from a variety of sources, for example, cells, cell-free preparations, chemical libraries, and natural product mixtures. Such compounds include, for instance, agonists, antagonists or inhibitors that may be natural or modified substrates, products, ligands, receptors, enzymes, etc., as the case may be, or may be structural or functional mimetics thereof (see Coligan et al., Current Protocols in Immunology 1(2):Chapter 5 (1991)).

Fusion proteins, such as those made from Fc portion and a polypeptide can also be used for high-throughput screening assays to identify antagonists of a target polypeptide (see D. Bennett et al., J Mol Recognition, 8:52-58 (1995); and K. Johanson et al., J Biol Chem, 270(16):9459-9471 (1995)).

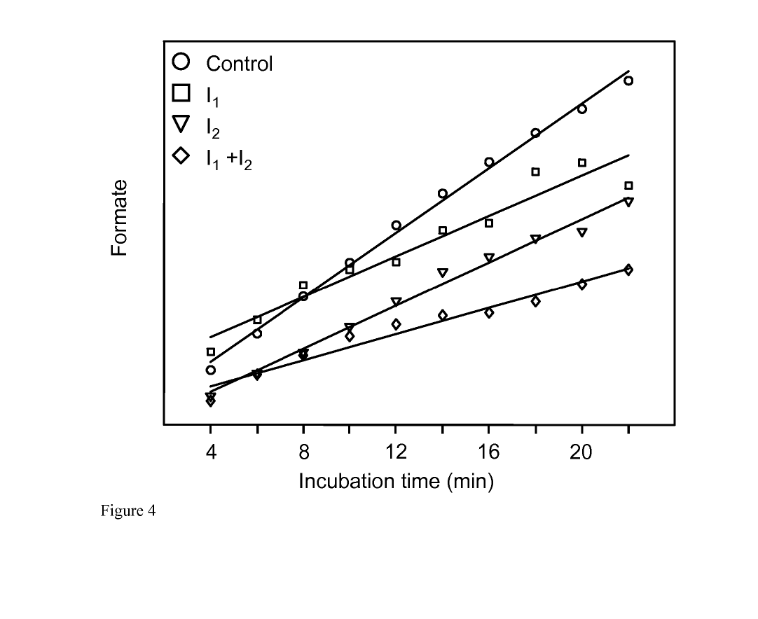

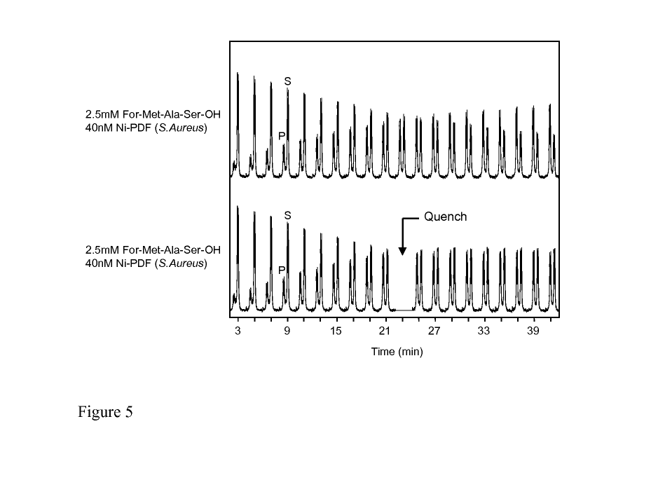

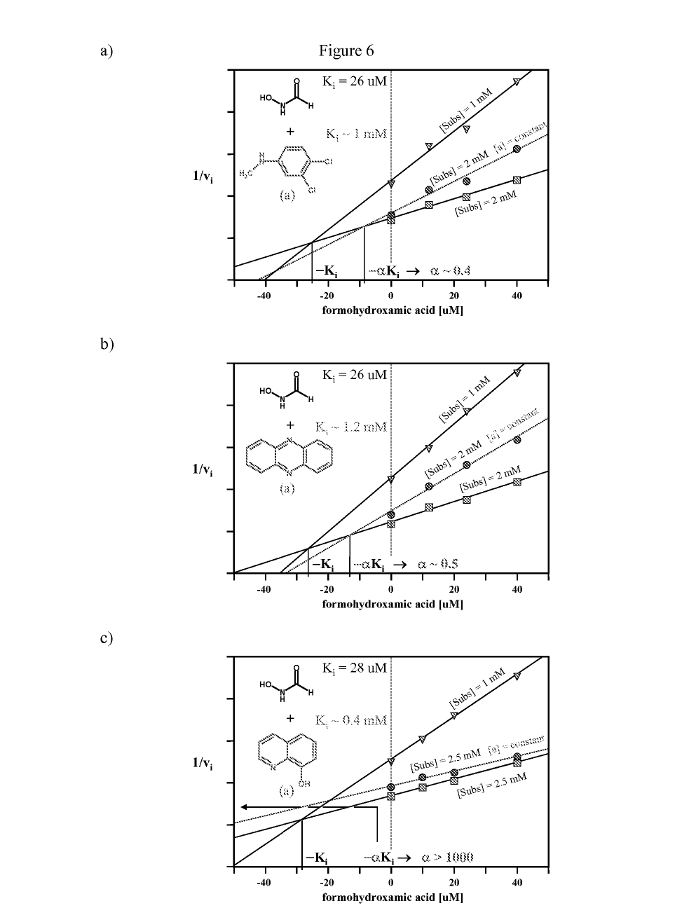

Certain methods of the invention are provided based on high throughput or parallel processing formats. One preferred high throughput embodiment is a methods wherein at least one chemical compound, but preferably a chemical mixture, is provided in a multiwell vessel loaded with target and ligand and/or substrate and/or product. The target may be already present in each well prior to loading of the ligand and/or substrate and/or product, or may be simultaneously or subsequently added with the ligand and/or substrate and/or product. Mutli-well plates are well known, commercially available, and useful with the methods of the invention. Once the target, at least one chemical compound, and a ligand and/or substrate and/or product is in the well, the reactions is allowed to progress for some desired time. After such time, the reaction is slowed or stopped (herein “quenched”) using a suitable method, readily determinable by a skilled artisan or known in the art for a given target. For example, quenching may be affected by heating, freezing, or by the addition of a chemical compound, as illustrated in FIG. 5. Common quenching compounds include, for example, alkylating agents, compounds that affect covalent modification of the target, target active site binding agents, antibodies, ligand or substrate mimetics, salts, organic solvents, EDTA, sodium azide, among others. Following quenching a reaction, the samples are loaded into an NMR device. This loading can be performed manually or using a robot. In preferred embodiments each of the wells in a multiwell format is loaded with one or more compounds, an invariant concentration of substrate and/or ligand and/or product, and an invariant concentration of target, most preferably an enzyme, either of protein or a ribozyme. In a preferred embodiment each reaction in the multiwell format is quenched simultaneously. This allows for each sample in each well to be loaded on an NMR device serially without having the undesirable effect of having reactions progress for different times. In another preferred embodiment, each or a series of reactions in the wells is quenched at differing time points. For instance, one or more reactions, each with a different chemical compound or compound mixture, is stopped at a first time, followed by one or more reactions, each with a different chemical compound or compound mixture, is stopped at a second time, etc., for as many time points as is desired. It is preferred that the mixed reagents in reactions stopped at one time point are provided in duplicate or further replicates and are be stopped at other time points. Two or more time points allow for one to calculate the effect of the chemical compound or mixture on the reaction over time. See, for example, FIG. 4, which also relates to determination of the interaction constant. Moreover, FIG. 6 illustrates a preferred embodiment of calculating the interaction constant using a graphical method with more measurements (See also, Yonetani, et a. (1982) Meth. Enzymol. 87: 500-509, and Yonetani, et a. (1964) Arch. Bioch. Biophys. 106: 243, for methods of calculating the interaction constant).

Ligands and/or substrates and/or products useful in methods of the invention include, for example, small organic molecules, peptides, polypeptides and antibodies that bind to a polynucleotide and/or polypeptide and thereby inhibit or extinguish its activity or expression. Ligands and/or substrates and/or prodyucts useful in methods of the invention include, for example, small organic molecules, a peptide, a polypeptide such as a closely related protein or antibody that binds the same sites on a target molecule thereby preventing the action or expression of target polypeptides and/or polynucleotides by excluding target polypeptides and/or polynucleotides from binding.

Ligands and/or substrates and/or products useful in methods of the invention include, for example, a small molecule that binds to and occupies the binding site of a target polypeptide thereby preventing binding to cellular binding molecules, such that normal biological activity is prevented. Examples of small molecules include but are not limited to small organic molecules, peptides or peptide-like molecules. Other potential antagonists include antisense molecules (see Okano, J. Neurochem. 56: 560 (1991); OLIGODEOXYNUCLEOTIDES AS ANTISENSE INHIBITORS OF GENE EXPRESSION, CRC Press, Boca Raton, Fla. (1988), for a description of these molecules). Preferred potential antagonists include compounds related to and variants of a target.

Other examples of potential polypeptide antagonists include antibodies or, in some cases, oligonucleotides or proteins which are closely related to the ligands, substrates, products, receptors, enzymes, etc., as the case may be, of a target, e.g., a fragment of the ligands, substrates, products, receptors, enzymes, etc.; or small molecules which bind to a target but do not elicit a response, so that the activity of the target is prevented.

### GLOSSARY

The following definitions are provided to facilitate understanding of certain terms used frequently herein.

“Antibody(ies)” as used herein includes polyclonal and monoclonal antibodies, chimeric, single chain, and humanized antibodies, as well as Fab fragments, including the products of an Fab or other immunoglobulin expression library.

“Bodily material(s) means any material derived from an individual or from an organism infecting, infesting or inhabiting an individual, including but not limited to, cells, tissues and waste, such as, bone, blood, serum, cerebrospinal fluid, semen, saliva, muscle, cartilage, organ tissue, skin, urine, stool or autopsy materials.

“Fusion protein(s)” refers to a protein encoded by two, often unrelated, fused genes or fragments thereof. In one example, EP-A-0464 discloses fusion proteins comprising various portions of constant region of immunoglobulin molecules together with another human protein or part thereof. In many cases, employing an immunoglobulin Fc region as a part of a fusion protein is advantageous for use in therapy and diagnosis resulting in, for example, improved pharmacokinetic properties [see, e.g., EP-A 0232262]. On the other hand, for some uses it would be desirable to be able to delete the Fc part after the fusion protein has been expressed, detected and purified.

“Individual(s)” means a multicellular eukaryote, including, but not limited to a metazoan, a mammal, an ovid, a bovid, a simian, a primate, and a human.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may contain many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, PROTEINS—STRUCTURE AND MOLECULAR PROPERTIES, 2nd Ed., T. E. Creighton, W. H. Freeman and Company, New York (1993) and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York (1983); Seifter et al., Meth. Enzymol. 182:626-646 (1990) and Rattan et al., Protein Synthesis: Posttranslational Modifications and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

“Variant(s)” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusion proteins and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. The present invention also includes include variants of each of the polypeptides of the invention, that is polypeptides that vary from the referents by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, 1-3, 1-2 or 1 amino acids are substituted, deleted, or added in any combination. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

### EXAMPLES

The examples below are carried out using standard techniques, which are well known and routine to those of skill in the art, except where otherwise described in detail. The examples are illustrative, but do not limit the invention.

### Example 1

### Deformylation by Peptide Deformylase

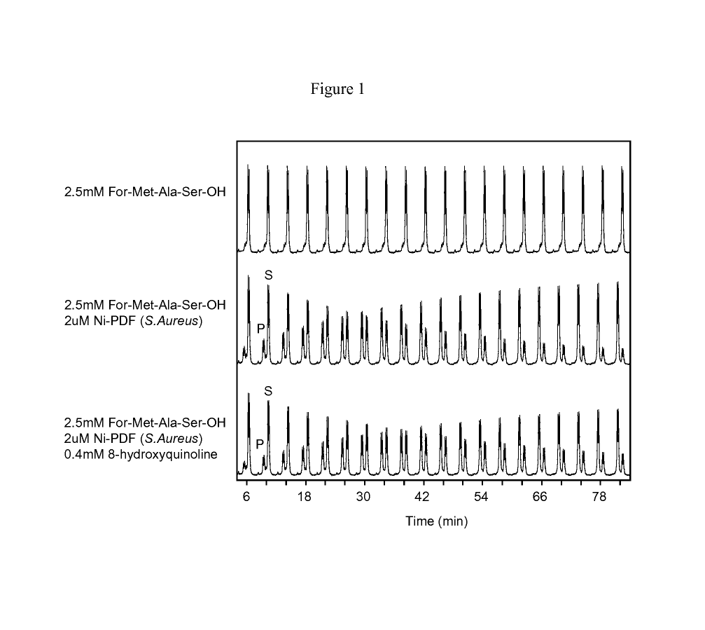

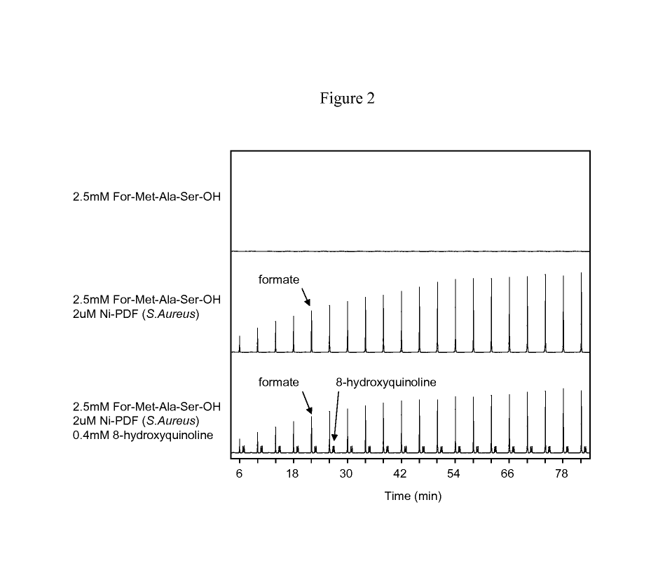

In a preferred embodiment of the present invention, catalytic efficiency is exploited in order to decrease the amount of target molecule required to detect binding of a compound to the target molecule. One may readily monitor changes in the conversion rate of a substrate into a product when the compound is present. FIGS. 1 and 2 illustrate such changes observed in the rate of deformylation of For-Met-Ala-Ser-OH by Peptide Deformylase (S.Aureus) when 8-hydroxyquinoline is present in the solution. With a catalytic efficiency in the range of 10⁵s⁻¹M⁻¹ for that substrate, it is possible to use a target concentration which is at least two orders of magnitude smaller than the concentration that would have been required for identifying 8-hydroxyquinoline as a ligand by the well-known NMR differential line broadening technique. Staphylococcus aureus Peptide Deformylase is described in U.S. patent application Ser. No. 08/911,844, filed Aug. 15, 1997, and and European Patent Application EP0879879 A2 and EP00879879A3. The methods of the example could also be adapted for use with Streptococcus pneumoniae pdf1 (U.S. patent application Ser. No. 08/991,023, filed Dec. 15, 1997 and European Patent Applications EP00863152A3 and EP00863152A2) or Streptococcus pneumoniae pdf2 (U.S. patent application Ser. No. 08/989,558, filed Dec. 12, 1997 and European Patent Application EP00863205A1).

What is claimed is:

1. A method using NMR for screening for ligands which exhibit synergistic effects on an enzyme in the presence of another ligand.

2. The method of claim 1 wherein the enzyme comprising more than two binding sites.

3. The method of claim 2 wherein the two binding sites are a substrate- and co-enzyme-binding site.

4. The method of claim 1 further comprising determining an interaction constant (α) comprising the steps of: a) exposing a substrate or product to an enzyme for one or more incubation times; b) generating a first spectrum that displays either a chemical shift in the first dimension or a chemical shifts in an other dimension of the substrate or product in step a) that has been exposed to the enzyme; c) mixing a substrate with the first ligand; d) exposing the substrate and the first ligand to the enzyme for one or more incubation times; e) generating a second spectrum that displays either a chemical shift in the first dimension or a chemical shift in the other dimension of substrate or product in step c) that has been exposed to the enzyme in step d) in the presence of the first ligand in step c); f) mixing the substrate or product with one or more chemical compounds; g) exposing the substrate or product and one or more chemical compounds to a target molecule for one or more incubation times; h) generating a third spectrum that displays either a chemical shift in the first dimension or a chemical shift in the other dimension of substrate or product in step f) that has been exposed to the enzyme in step g) in the presence of the one or more chemical compounds in step f); i) mixing substrate or product with first ligand and one or more chemical compounds; j) exposing the substrate or product, the first ligand and the one or more chemical compounds to the target molecule for one or more incubation times; k) generating a fourth spectrum that displays either a chemical shift in the first dimension or a chemical shift in the other dimension of the substrate or product in step i) that has been exposed to the enzyme in step j) in the presence of the first ligand and the one or more chemical compounds in step f); l) determining a conversion rate or conversion rates of each substrate or product from each spectrum of steps b), e), h) and k); and deriving an interaction constant (α) from a steady-state rate equation.

5. The method of claim 4 wherein the rate is determined by the following equation:
$$v = {V}_{m} / [ 1 + ( \frac{{K}_{M}}{S} ) ( 1 + \frac{{I}_{1}}{{K}_{EI 1}} + \frac{{I}_{2}}{{K}_{EI 2}} + \frac{{I}_{1} {I}_{2}}{α {K}_{EI 1} {K}_{EI 2}} ) ]$$
wherein S, I₁ and I₂ are the substrate, inhibitor I₁ and inhibitor I₂ concentrations, respectively.
